(19) United States
(12) Patent Application Publication
(10) Pub. No.: US 2022/0087614 A1
(43) Pub. Date: Mar. 24, 2022

(54) NON-INVASIVE TYPE ECG MONITORING DEVICE AND METHOD

(71) Applicant: Industry-Academic Cooperation Foundation, Yonsei University, Seoul (KR)

(72) Inventors: Jeong Gil KO, Yongin-si (KR); Jae Yeon PARK, Hwaseong-si (KR)

(73) Assignee: Industry-Academic Cooperation Foundation Yonsei University

(21) Appl. No.: 17/376,395

(22) Filed: Jul. 15, 2021

(30) Foreign Application Priority Data
Sep. 18, 2020 (KR) 10-2020-0120417

(51) Int. Cl. A61B 5/00; A61B 5/318; A61B 5/024

(52) U.S. Cl. CPC A61B 5/721; A61B 5/7225; A61B 5/024; A61B 5/318; A61B 5/7267

(57) ABSTRACT

An electrocardiogram (ECG) monitoring device includes a vibration sensor unit configured to detect a vibration transferred through an instrument in a non-contact and non-invasive manner to acquire a vibration signal, the vibration sensor unit including at least one vibration sensor attached to an instrument where an object under observation is located, a filter unit configured to receive the vibration signal, filter the vibration signal to remove a predetermined frequency band, and extract a seismocardiography (SCG) signal caused by a heart vibration of the subject under observation; and an ECG waveform acquisition unit configured to estimate a pattern of the SCG signal and generate an ECG signal of a pattern corresponding to the pattern of the SCG signal, the ECG waveform acquisition unit including a pre-trained artificial neural network.

### CROSS-REFERENCE TO RELATED APPLICATION

This application claims priority to and the benefit of Korean Patent Application No. 2020-0120417, filed on Sep. 18, 2020, the disclosure of which is incorporated herein by reference in its entirety.

### BACKGROUND

### 1. Field of the Invention

The present disclosure relates to an electrocardiogram monitoring device and method and, more particularly, to a non-invasive electrocardiogram monitoring device and method using a small vibration sensor.

### 2. Discussion of Related Art

In general, heart-related diseases such as arrhythmia and myocardial infarction are diagnosed using an electrocardiogram (ECG) waveform. In order to accurately diagnose heart diseases at an early stage, continuous ECG waveform analysis is required rather than short-term fragmentary measurements. In particular, in the case of high-risk patients, sudden death from heart-related diseases occurs even in large hospitals during night hours while they are sleeping in addition to during daytime hours when continuous medical observation is possible.

The Holter test currently used for continuous ECG waveform monitoring includes attaching multiple ECG monitoring sensors to the body of a subject under observation and then observing a patient every 24-48 hours. However, since the sensors are attached to sleeping subjects to continuously collect biometric information, the Holter test equipment disrupts their sleep patterns due to the inconvenience of wearing, the mixing of dynamic noise, and the like. This makes it practically difficult to stably and continuously collect data from sleeping patients to be observed.

Also, ECG measuring equipment such as a Holter monitor is very expensive and is difficult to possess in large quantities even in large hospitals. Therefore, it is very difficult for patients who have been discharged from a hospital or people at risk for heart disease to use ECG measuring equipment at home. Thus, there is a problem in that emergency measures cannot be taken if a heart abnormality occurs while a patient is sleeping at home.

To compensate for this problem, some wearable devices have recently been implemented to measure the heart rate, but measuring a heart rate is not sufficient to detect various heart conditions such as arrhythmia or myocardial infarction.

Therefore, it is necessary to secure a technology that can continuously extract ECG waveforms during sleep at low cost by using non-invasive sensor equipment without attaching additional sensors to the body of a subject under observation.

### SUMMARY

The present disclosure is directed to providing an electrocardiogram monitoring device and method capable of monitoring the electrocardiogram of a subject under observation at low cost.

The present disclosure is also directed to providing an electrocardiogram monitoring device and method capable of accurately monitoring the electrocardiogram in a non-contact and non-invasive manner without causing inconvenience to a subject under observation.

According to an aspect of the present disclosure, there is provided an electrocardiogram (ECG) monitoring device including a vibration sensor unit configured to detect a vibration transferred through an instrument in a non-contact and non-invasive manner to acquire a vibration signal, the vibration sensor unit including at least one vibration sensor attached to the instrument where an object under observation is located; a filter unit configured to receive the vibration signal, filter the vibration signal to remove a predetermined frequency band, and extract a seismocardiography (SCG) signal caused by a heart vibration of the subject under observation; and an ECG waveform acquisition unit configured to estimate a pattern of the SCG signal and generate an ECG signal of a pattern corresponding to the pattern of the SCG signal, the ECG waveform acquisition unit including pre-trained artificial neural network.

The filter unit may include a first filter unit implemented as a low pass filter and configured to receive the vibration signal and filter the vibration signal to remove a frequency band of more than a first predetermined frequency, and a second filter unit implemented as a high pass filter and configured to receive the filtered signal from the first filter unit and filter the received signal to remove a frequency band of less than a second predetermined frequency.

The filter unit may further include a noise analysis unit configured to set the first frequency and the second frequency according to at least one of intrinsic noise of the at least one vibration sensor or noise caused by an ambient environment of the instrument and provide the first frequency and the second frequency to the first filter unit and the second filter unit.

The noise analysis unit may set the first frequency and the second frequency according to a frequency of the vibration signal applied at a predetermined time when the subject under observation is not located in the instrument or a frequency detected from the vibration signal while the strength of a signal of a specific frequency band is less than or equal to a reference strength.

A frequency band of the intrinsic noise of the at least one vibration sensor may be measured and stored in advance, and the noise analysis unit may set the first frequency and the second frequency according to the stored frequency band of the intrinsic noise.

The at least one vibration sensor may be implemented as a geophone, and the noise analysis unit may set the first frequency to 30 Hz and set the second frequency to 5 Hz.

The ECG waveform acquisition unit may include a sampling unit configured to sample the SCG signal at a predetermined sampling rate to convert the SCG signal into SCG data, and an ECG pattern estimation unit implemented as a pre-trained Bidirectional Long/Short-Term Memory (Bi-LSTM) neural network and configured to estimate a pattern change over time of the SCG data, which is time-series data, and acquire ECG data having a pattern corresponding to the estimated pattern change.

The ECG pattern estimation unit may include a first Bi-LSTM layer including a forward layer and a backward layer each including multiple LSTM cells that receive SCG data included in a corresponding area while moving a sliding window of a predetermined size in chronological order with respect to the SCG data and extract features of the SCG data in a pre-trained manner and an activation layer configured to normalize outputs of the forward layer and the backward layer and output the normalized outputs, a second Bi-LSTM layer including a forward layer and a backward layer each including multiple LSTM cells that receive an output of the first Bi-LSTM layer and extract features of the output of the first Bi-LSTM layer in a pre-trained manner and an activation layer configured to normalize outputs of the forward layer and the backward layer and output the normalized outputs, and a regression layer configured to classify the output of the second Bi-LSTM layer as a corresponding value in a pre-trained manner and acquire the ECG data, the regression layer including multiple fully connected layers.

The ECG pattern estimation unit may be trained by calculating, among learning data including multiple pieces of SCG data pre-acquired using a separate ECG measurement device and multiple pieces of ECG data mapped to the multiple pieces of SCG data, an error between ECG data acquired by receiving the multiple pieces of SCG data as an input and ECG data mapped in the learning data and backpropagating the calculated error.

The ECG waveform acquisition unit may further include a personal data storage unit configured to store personal data including multiple pieces of SCG data pre-acquired for the subject under observation using a separate ECG measurement device and multiple pieces of ECG data mapped to the multiple pieces of SCG data, and the ECG pattern estimation unit may be additionally trained using the personal data stored in the personal data storage unit.

The ECG waveform acquisition unit may further include an ECG waveform analysis unit configured to receive the ECG data and convert the ECG data into an analog signal to acquire the ECG signal or analyze the ECG data to extract multiple predetermined clinical indicators.

According to another aspect of the present disclosure, there is provided an electrocardiogram (ECG) monitoring method including detecting a vibration transferred through an instrument in a non-contact and non-invasive manner to acquire a vibration signal using at least one vibration sensor attached to the instrument where an object under observation is located; receiving the vibration signal, filtering the received vibration signal to remove a predetermined frequency band, and extracting a seismocardiography (SCG) signal caused by a heart vibration of the subject under observation; and estimating a pattern of the SCG signal and generating an ECG signal of a pattern corresponding to the pattern of the SCG signal using a pre-trained artificial neural network.

### BRIEF DESCRIPTION OF THE DRAWINGS

The above and other objects, features and advantages of the present disclosure will become more apparent to those of ordinary skill in the art by describing exemplary embodiments thereof in detail with reference to the accompanying drawings, in which:

FIG. 1 is a diagram schematically showing an electrocardiogram monitoring device according to an embodiment of the present disclosure;

FIG. 2 is a diagram illustrating the operation of each component of the monitoring device of FIG. 1;

FIG. 3 shows the frequency distribution of seismocardiography (SCG) signal waveforms measured from multiple subjects under observation;

FIG. 4 shows a result of measuring intrinsic noise of a vibration sensor included in a vibration sensor unit of FIG. 1;

FIG. 5 shows examples of electrocardiogram and SCG signal waveforms according to heart activity;

FIG. 6 shows an example of the detailed configuration of an electrocardiogram waveform acquisition unit of FIG. 1;

FIG. 7 shows a vibration signal waveform acquired by a vibration sensor unit of FIG. 1, a SCG signal waveform filtered by a filter unit, and an electrocardiogram signal waveform extracted by an electrocardiogram waveform acquisition unit;

FIG. 8 is a diagram illustrating important clinical indicators corresponding to electrocardiogram signal waveforms; and

FIG. 9 illustrates an electrocardiogram monitoring method according to an embodiment of the present disclosure.

### DETAILED DESCRIPTION OF EXEMPLARY EMBODIMENTS

For a better understanding of the invention, its operating advantages and the specific objects attained by its uses, reference should be made to the accompanying drawings and descriptive matter in which preferred embodiments of the disclosure are illustrated.

Hereinafter, the present disclosure will be described in detail by explaining exemplary embodiments of the present disclosure with reference to the accompanying drawings. The exemplary embodiments may, however, be embodied in many different forms and should not be construed as being limited to the embodiments set forth herein. In addition, in order to clearly describe the present disclosure, parts irrelevant to the description will be omitted, and the same reference numerals in the drawings denote the same members.

Furthermore, when a part is referred to as “including” elements, it should be understood that it can include only those elements or other elements as well as those elements unless specifically described otherwise. Also, terms such as “unit,” “-er,” “-or,” “module,” and “block” used herein refer to an element for performing at least one function or operation and may be implemented with hardware, software, or a combination thereof.

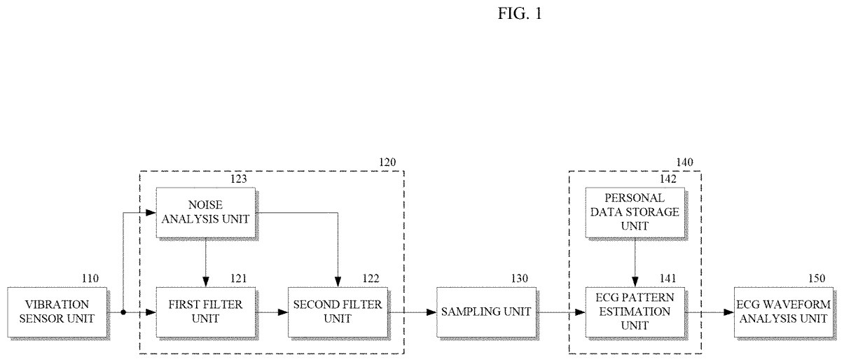

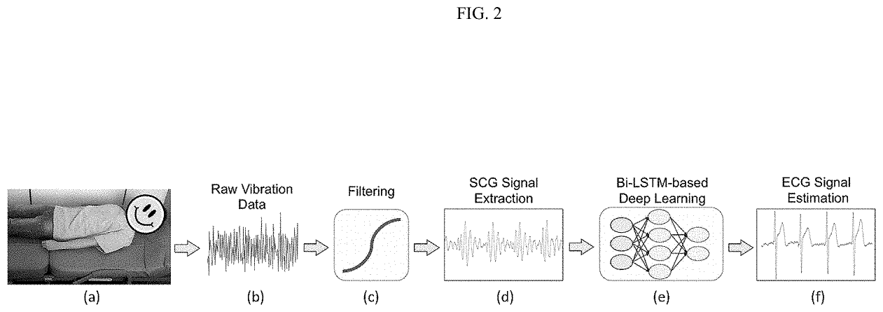

FIG. 1 is a diagram schematically showing an electrocardiogram (ECG) monitoring device according to an embodiment of the present disclosure, and FIG. 2 is a diagram illustrating the operation of each component of the monitoring device of FIG. 1

Referring to FIG. 1, the ECG monitoring device according to an embodiment may include a vibration sensor unit 110, a filter unit 120, a sampling unit 130, an ECG waveform acquisition unit 140, and an ECG waveform analysis unit 150.

First, the vibration sensor unit 110 is implemented as a small vibration sensor to detect vibrations generated by a subject under observation. In an embodiment, as an example, the vibration sensor unit 110 may be implemented as a geophone, which is a small vibration sensor. A geophone may include a coil, a magnet, and two springs disposed at upper and lower ends to measure the amount of vibration from inertial mass applied to the springs. In particular, an SM-24 geophone sensor, which is inexpensive, compact, and widely used to detect earthquakes in the surrounding environment, may be used herein. An SM-24 geophone sensor may provide a reliable sensitivity of 28.8 V/m/s in the frequency range of 0.5 to 50 Hz at low cost.

In addition, the vibration sensor unit 110 does not come into direct contact with the body of a subject under observation, unlike conventional ECG measurement equipment.

A normal human heart repeats contraction and relaxation in the process of supplying blood, and small vibrations are generated in this process. That is, a vibration is generated according to the seismocardiography (SCG) of the subject under observation. In the past, it was difficult to detect a weak vibration such as the SCG of a subject under observation who was not in direct contact with the vibration sensor. However, recently, along with the improvement of sensitivity of a small vibration sensor such as a geophone, it has become possible to detect the SCG of a subject under observation only by indirect contact without direct contact with the subject under observation.

Therefore, according to an embodiment, the vibration sensor unit 110 is attached to various pieces of furniture, such as a bed, a chair, etc., where a subject under observation is located and is configured to detect vibrations only by indirect contact with the subject under observation. The location where the vibration sensor unit 110 is placed is not limited, but it is assumed here that, as an example, the vibration sensor unit 110 is installed on a bed on which a subject under observation is lying, as shown in FIG. 2A. The vibration sensor unit 110 may be placed on an upper panel of a mattress to effectively detect a heart vibration of the subject under observation. In particular, the vibration sensor unit 110 may be placed near the left shoulder of the subject under observation. Therefore, the ECG monitoring device according to an embodiment may easily monitor the ECG of the subject under observation during sleep.

The vibration sensor unit 110 may send a vibration signal corresponding to the sensed vibration to the filter unit 120 in a wired or wireless manner.

The filter unit 120 acquires an SCG signal by filtering out noise from the vibration signal applied from the vibration sensor unit 110. In the vibration signal acquired from the vibration sensor unit 110, various kinds of noise as well as a vibration generated in the heart of a subject under observation are contained. Even when the vibration sensor unit 110 is in direct contact with the subject under observation, various vibrations such as a vibration due to movement or respiration of the subject under observation as well as the heart vibration of the subject under observation may also be detected and contained as noise in the vibration sensor unit 110.

In particular, in this embodiment, the vibration sensor unit 110 detects a vibration through indirect contact without being in direct contact with or invasive to the subject under observation. Therefore, compared to the direct contact method, various types of noise in addition to a vibration generated by the heartbeat of the subject under observation may be contained in the acquired vibration signal. As an example, when a vibration sensor is attached to a bed, vibration of a mattress, vibration generated in the surrounding environment due to a person walking in the vicinity, etc. may be further included as noise. In addition, in some cases, inertial noise of the vibration sensor itself may also be included.

Accordingly, the filter unit 120 acquires an SCG signal by filtering out noise components excluding heart vibration components from the vibration signal applied from the vibration sensor unit 110.

The filter unit 120 may be configured to receive a vibration signal applied from the vibration sensor unit 110, pass only a signal of a predetermined frequency band out of the applied vibration signal, and block signals of the other frequency bands to remove noise included in the vibration signal.

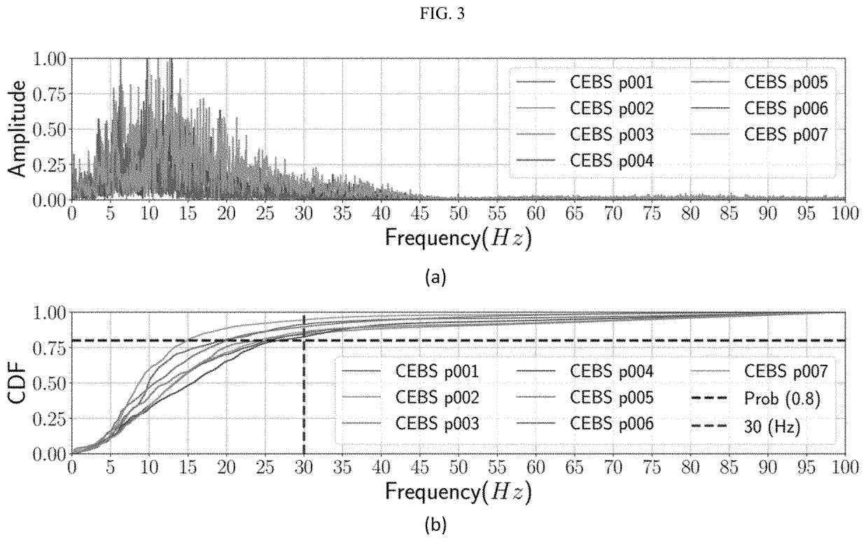

FIG. 3 shows the frequency distribution of SCG signal waveforms measured from multiple subjects under observation.

FIG. 3 shows a result of measuring SCG signals from multiple subjects under observation. FIG. 3A shows the frequency signal of the SCG signal obtained by performing Fast Fourier Transform on SCG signals measured using conventional measurement equipment, and FIG. 3B shows the cumulative distribution according to the frequency of the frequency signal of FIG. 3A. As shown in FIGS. 3A and 3B, it can be seen that an SCG signal contains most of the signal components in a frequency band of 0 to 45 Hz regardless of the subject under observation. Also, even when an SCG signal is acquired using measurement equipment, noise such as breathing of a subject under observation is contained, and thus in general, signal components in the band of 5 Hz or less are treated as noise.

Accordingly, the filter unit 120 may be implemented as a band pass filter (BPF) that leaves only signal components in a frequency band of 5 Hz to 45 Hz out of the applied vibration signal and blocks the remaining signal components.

However, in the case of a BPF, filtering is performed so that both frequency bands are symmetrical with respect to a center frequency fc. Therefore, it is not easy to apply a BPF when noise is asymmetrically distributed in several frequency bands.

Accordingly, in an embodiment, the filter unit 120 may include a first filter unit 121 and a second filter unit 122. Here, the first filter unit 121 may be implemented as a low pass filter (LPF), and the second filter unit 122 may be implemented as a high pass filter (HPF), or vice versa.

That is, the filter unit 120 is configured to filter out noise by combining an LPF and an HPF. Here, the LPF and the HPF included in the first filter unit 121 and the second filter unit 122 may be implemented to perform filtering using Equation 1 and Equation 2, respectively.

$$\begin{matrix}L ( s ) = \frac{{ω}_{0}^{2} \cdot {H}_{0}}{\frac{{ω}_{0}}{Q} s + {ω}_{0}^{2} + {s}^{2}} & [ Equation 1 ] \\ H ( s ) = \frac{{s}^{2} \cdot {H}_{0}}{\frac{{ω}_{0}}{Q} s + {ω}_{0}^{2} + {s}^{2}} & [ Equation 2 ]\end{matrix}$$

Here, s is an s-domain frequency signal, Q is a quality factor, and ω₀ is a cutoff angular frequency (ω₀=2πf_{cut}) corresponding to a cutoff frequency (f_{cut}).

As described above, in order for the filter unit 120 to pass only signals in a frequency band of 5 Hz to 45 Hz, when the first filter unit 121 is implemented as an LPF and the second filter unit 122 is an HPF, the cutoff frequency f_{cut} of the LPF of Equation 1 may be set to 45 Hz, and the cutoff frequency f_{cut} of the HPF of Equation 2 is 5 Hz may be set to 5 Hz. That is, it is possible to filter out a low frequency band of less than 3 Hz and a high frequency band of 35 Hz or more.

However, as described above, the vibration sensor unit 110 in the ECG monitoring device according to an embodiment detects vibrations through indirect contact without being in direct contact with or invasive to a subject under observation. Therefore, vibrations caused by the surrounding environment may also be detected and contained as noise. In addition, since the vibration sensor unit 110 is used instead of an ECG measurement device, intrinsic noise, such as inertial noise, may be generated in the vibration sensor included in the vibration sensor unit 110. Accordingly, the filter unit 120 may be implemented to adaptively perform filtering by varying the filtering frequency according to the vibration sensor and the surrounding environment in consideration of noise caused by the vibration sensor and noise caused by the surrounding environment.

Therefore, according to an embodiment, the filter unit 120 may further include a noise analysis unit 123 in which a frequency band to be filtered by the filter unit 120 is set and stored so as to perform adaptive filtering.

First, a frequency component of the intrinsic noise of the vibration sensor included in the vibration sensor unit 110 may be measured and stored in advance in the noise analysis unit 123.

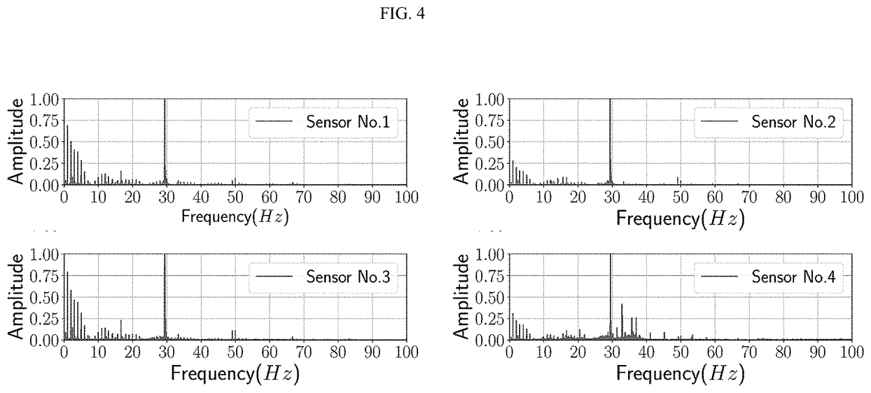

FIG. 4 shows a result of measuring intrinsic noise of a vibration sensor included in a vibration sensor unit of FIG. 1.

FIG. 4 shows a result of measuring the inertial noise of an SM-24 geophone sensor itself. Also in FIG. 4, as a result of converting the measured noise signal into a frequency domain signal through fast Fourier transform, FIGS. 4A to 4D show results of converting noise measured from four different SM-24 geophone sensors into the frequency domain.

Referring to FIGS. 4A to 4D, the four SM-24 geophone sensors all show similar noise signal patterns. A great deal of noise is mainly generated in a frequency band of 0 Hz to 5 Hz, and high peak noise is generated at a frequency of 30 Hz.

This means that when the filter unit 120 filters out signals in a frequency band of less than 5 Hz and a frequency band of more than 30 Hz, most of the intrinsic noise of the SM-24 geophone sensor can be removed. However, a frequency band of less than 5 Hz and a frequency band of more than 30 Hz are noise bands of the SM-24 geophone sensor. When the vibration sensor unit 110 is implemented as a vibration sensor other than the SM-24 geophone sensor, the noise bands may be changed.

In addition, frequency components of noise generated, due to the surrounding environment, from a signal detected while a subject under observation is not located on furniture, such as beds or sofas to which the vibration sensor unit 110 is attached, may be analyzed and stored in advance in the noise analysis unit 123. As an example, the noise analysis unit 123 may analyze ambient environmental noise when a vibration signal is applied in a predetermined period of time or when a signal of a specific frequency band is not included in an applied vibration signal. Here, the noise analysis unit 123 may analyze ambient environmental noise by analyzing a vibration signal applied in the morning or during the daytime when the subject under observation does not enter into a sleeping state. Also, when the object under observation is not located on a bed to which the vibration sensor unit 110 is attached, the noise analysis unit 123 may detect a vibration signal in which the strength of a signal with a frequency band of 5 Hz to 35 Hz corresponding to an SCG signal is less than or equal to a predetermined reference strength and may analyze an ambient environmental noise.

However, since generally, most of the ambient environmental noise also has a predetermined frequency band (e.g., less than 5 Hz), a frequency band for filtering out the intrinsic noise of the vibration sensor and the ambient environmental noise may be input and stored in advance in the noise analysis unit 123.

In this case, the noise analysis unit 123 should allow a frequency band to be set to filter out noise without filtering the SCG signal as much as possible in order to prevent an ECG signal from being inaccurately acquired due to the filtering of an SCG signal component to be acquired

Referring back to FIG. 3, the SCG signal includes approximately 80% or more of the entire signal component in a frequency band of 5 Hz to 30 Hz. Therefore, the noise analysis unit 123 may be allowed to remove noise from the vibration signal while preserving SCG components by setting a filtering frequency band so that the first filter unit 121 and the second filter unit 122 can pass a frequency band of 5 Hz to 30 Hz and remove signals of the other frequency bands.

When an SCG signal is acquired, as shown in FIG. 2D, from a signal obtained by filtering the vibration signal through the filter unit 120, the sampling unit 130 receives the acquired SCG signal, samples the received SCG signal to perform digital conversion, and acquires SCG data. The sampling unit 130 may acquire the SCG data by sampling the SCG signal at a sampling rate of a predetermined frequency (e.g., 250 Hz) higher than the filtering frequency of the filter unit 120. In this case, the sampling unit 130 may normalize the sampled SCG signal to a predetermined range (here, e.g., [−1:1]).

Here, the sampling unit 130 is separately provided for convenience of description but may be included in the ECG waveform acquisition unit 140.

The ECG waveform acquisition unit 140 extracts ECG data from the SCG data.

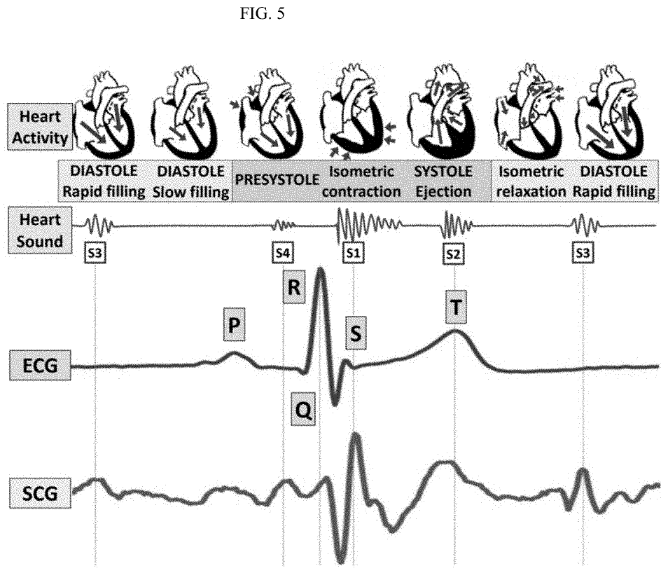

FIG. 5 shows examples of ECG and SCG signal waveforms according to heart activity.

Referring to FIG. 5, the SCG signal is a signal obtained by detecting a vibration generated in the heart of a subject under observation and is not an ECG signal. Although the SCG signal is also a signal useful for analyzing the heart activity of the subject under observation, there is a limit to accurately understanding the heart activity of the subject under observation only with the pattern of the SCG signal.

However, as shown in FIG. 5, it is well known that a correlation between the waveform of the SCG signal and the waveform of the ECG signal is high. Both of an SCG signal and an ECG signal are time-series signals collected from a human heart, and the SCG signal represents a vibration due to the periodic motion and blood flow of the heart, and the ECG signal represents a corresponding electrical signal. This is because, from a clinical point of view, the electrical activity of the heart causes periodic depolarization and repolarization, which induces periodic cardiac muscle contraction, cardiac muscle relaxation, and blood flow.

In FIG. 5, peaks P, Q, R, S, and T of the ECG signal are generated by the electrical activity of the heart, and four core heart sounds S1 to S4 are also detected in the SCG signal. Specifically, the first heart sound S1 is the closing sound of the mitral valve, which is a valve between the left atrium and the left ventricle, and the second heart sound S2 is the closing sound of the aortic valve, which is a half-moon-shaped valve connecting the ventricles. In addition, the third heart sound S3 is an exogenous sound due to rapid filling of the aorta, and the fourth heart sound S4 is a gallop rhythm just before artery contraction, and all of the sounds are generated by the electrical activity of the heart.

However, due to the diversity of an SCG signal pattern and the complexity of noise, the exact relationship between the SCG signal pattern and the ECG signal pattern has not yet been elucidated. For this reason, even when an SCG signal was acquired, an ECG signal had to be monitored separately, and as described above, ECG had to be measured using an expensive direct-attached device as in the Holter test scheme.

Accordingly, as shown in FIG. 2E, the ECG waveform acquisition unit 140 according to an embodiment enables ECG data to be extracted from the digitally converted SCG data using a pre-trained artificial neural network.

The ECG waveform acquisition unit 140 may include an ECG pattern estimation unit 141. Here, the ECG pattern estimation unit 141 may be implemented as a pre-trained artificial neural network. In particular, the ECG pattern estimation unit 141 may be implemented, for example, as a Bidirectional Long/Short-Term Memory (LSTM) (hereinafter referred to as Bi-LSTM).

Bi-LSTM includes, based on time, a forward layer including multiple LSTM cells that transmit and process information in the forward direction and a backward layer including multiple LSTM cells that transmit and process information in the backward direction, and thus it is possible to facilitate the extraction of ECG patterns that appear differently depending on individual subjects under observation.

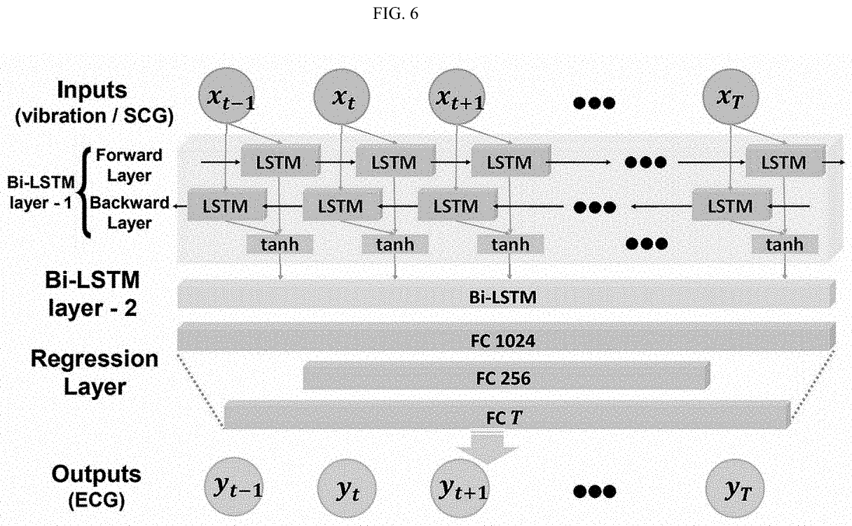

FIG. 6 shows an example of the detailed configuration of an electrocardiogram waveform acquisition unit of FIG. 1.

Referring to FIG. 6, the ECG pattern estimation unit 141 according to an embodiment may include two Bi-LSTM layers (Bi-LSTM layer-1 and Bi-LSTM layer-2) and a regression layer.

Each of the two Bi-LSTM layers (Bi-LSTM layer-1 and Bi-LSTM layer-2) may include a forward layer and a backward layer, each of which includes multiple LSTM cells, and an activation layer.

When the ECG pattern estimation unit 141 uses an LSTM layer including only a forward layer instead of a Bi-LSTM layer, while an applied SCG signal passes through multiple LSTM cells of the LSTM layer, an initial sequence xₜ₋₁ in the SCG signal may be treated as less important than a subsequent sequence x_{T}. On the other hand, since the Bi-LSTM layer includes both a forward layer and a backward layer, forward sequences (xₜ₋₁, xₜ, xₜ₊₁, . . . xT) and backward sequences (x_{T}, x_{T-1}) may be processed in the SCG signal equally in terms of time. That is, the Bi-LSTM layer ensures that all regions of the applied SCG signal have equal importance.

Also, the activation layer may be implemented as a hyperbolic tangent function (tan h) so that the outputs of the two Bi-LSTM layers are normalized to a predetermined range (here, e.g., [−1:1]) and output like the input SCG data.

The ECG pattern estimation unit 141 moves a sliding window in chronological order with respect to the SCG data transferred from the sampling unit 130, extracts SCG data of a corresponding area, and transfers the SCG data to the forward layer and the backward layer of the first Bi-LSTM layer (Bi-LSTM layer-1), which is one of the two Bi-LSTM layers (Bi-LSTM layer-1 and Bi-LSTM layer-2).

In this case, the size of the sliding window should be set to a size corresponding to two or more periods of the SCG signal. This allows the SCG data corresponding to two periods in the SCG signal to be simultaneously applied to the first Bi-LSTM layer (Bi-LSTM layer-1) so that the relationship between the SCG signal pattern and the ECG signal pattern corresponding to the ECG data can be accurately estimated. In particular, in order to accurately estimate the time relationship between two consecutive periods of the ECG signal, such as an RR interval, which is one of the important indicators in the ECG signal, the size of the sliding window should be set to a size corresponding to two or more periods of the SCG signal.

Therefore, according to an embodiment, it is assumed that the size of the sliding window is set to a size including SCG data corresponding to a length more than twice (e.g., three times in this case) a specified sampling rate.

Also, the ECG pattern estimation unit 141 includes two Bi-LSTM layers (Bi-LSTM layer-1 and Bi-LSTM layer-2), and thus the second Bi-LSTM layer (Bi-LSTM layer-2) receives the output of the first Bi-LSTM layer (Bi-LSTM layer-1) so as to allow various patterns of nonlinear relationships between the ECG signal and the SCG signal to be accurately mapped. In particular, this is to reflect the relationship between the SCG data extracted from the previous sliding window and the SCG data extracted from a subsequent sliding window.

That is, since the two Bi-LSTM layers (Bi-LSTM layer-1 and Bi-LSTM layer-2) are arranged in a stacked structure, the ECG pattern estimation unit 141 may clearly estimate a change pattern relationship of a heartbeat signal in a wide time interval.

The features of the SCG data extracted through the first and second Bi-LSTM layers (Bi-LSTM layer-1 and Bi-LSTM layer-2) including multiple fully-connected layers FC of the regression layer are classified to acquire ECG data at a specific time point T. Since the sliding window extracts and transfers SCG data of a specified size from SCG data continuously applied over time, the regression layer may continuously acquire ECG data corresponding to each time point. In this case, the multiple fully-connected layers FC may be connected in series to have a structure in which the size is gradually reduced and may be finally configured to output ECG data at a specific time point T.

Here, the ECG pattern estimation unit 141 may be trained using multiple pieces of learning data acquired by mapping the ECG data and the SCG data measured using separate measurement equipment, etc. in advance. The ECG pattern estimation unit 141 receives the SCG data of the learning data that is measured and acquired in advance as an input and outputs ECG data corresponding to the received SCG data. When the corresponding ECG data is output, the ECG pattern estimation unit 141, which is implemented as an artificial neural network, may be trained by calculating an error between the corresponding ECG data and the ECG data mapped to the SCG data input as the training data, propagating the calculated error back to the ECG pattern estimation unit 141, and repeating this process until the error is less than or equal to a predetermined reference error.

That is, the ECG pattern estimation unit 141 is trained using the ECG data and the SCG data of the learning data and thus may output ECG data corresponding to the SCG data applied from the sampling unit 130, as shown in FIG. 2F.

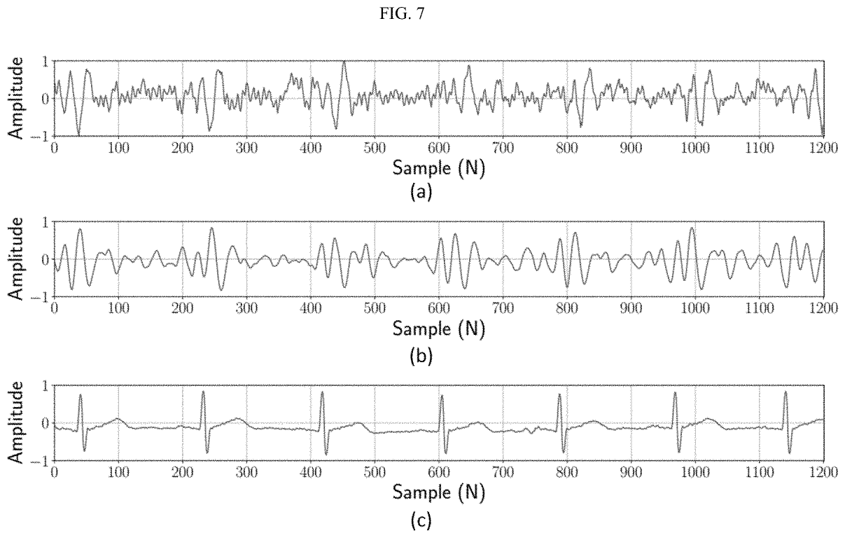

FIG. 7 shows a vibration signal waveform acquired by a vibration sensor unit of FIG. 1, an SCG signal waveform filtered by a filter unit, and an electrocardiogram signal waveform extracted by an electrocardiogram waveform acquisition unit.

FIG. 7A shows a vibration signal acquired by the vibration sensor unit 110, FIG. 7B shows an SCG signal filtered by the filter unit 120, and FIG. 7C shows an ECG signal corresponding to ECG data extracted by the ECG pattern estimation unit 141.

As shown in FIG. 7A, a vibration signal acquired by the vibration sensor unit 110 contains a significant amount of noise. However, noise is removed by the filter unit 120 from the SCG signal of FIG. 7B, and thus it can be seen that the pattern of the waveform has become clearer. Also, it can be seen that an ECG signal extracted from the SCG signal is extracted as a very regular waveform to estimate the heart condition of the subject under observation. That is, a medical practitioner can easily determine the heart condition of the subject under observation.

Meanwhile, SCG data and ECG data measured for each subject under observation may be stored in a personal data storage unit 142. As described above, the ECG pattern estimation unit 141, which is implemented as an artificial neural network, may be trained using a large amount of learning data. However, the patterns of the SCG signal and the ECG signal vary for each person. Therefore, when the ECG pattern estimation unit 141 can perform training based on an SCG signal and an ECG signal acquired, in advance, from a subject under observation, the ECG pattern estimation unit 141 may extract more accurate ECG data from the SCG data.

Also, when a patient with a heart disease is a subject under observation, SCG data and ECG data already obtained using an ECG measurement device may exist in a hospital in most cases. Therefore, when the ECG pattern estimation unit 141 is trained by using SCG data and ECG data, as learning data, that are already acquired using the ECG measuring equipment, an ECG pattern estimation unit 141 personalized for a specific subject under observation may be acquired.

Therefore, according to the present disclosure, the ECG waveform acquisition unit 140 may further include a personal data storage unit 142 in which the ECG data and the SCG data acquired using the ECG measurement device are stored. Here, the personal data storage unit 142, which is a component for training the ECG pattern estimation unit 141, may be removed after the training is completed.

However, since a large amount of learning data is required to normally train the ECG pattern estimation unit 141, it is not possible to normally train the ECG pattern estimation unit only with the SCG signal and ECG signal collected for a specific personal subject under observation. Therefore, according to the present disclosure, the ECG waveform acquisition unit 140 is pre-trained based on a large amount of learning data and then additionally trained with the learning data acquired for a specific personal subject under observation, and thus it is possible to personalize the ECG pattern estimation unit 141.

The ECG waveform analysis unit 150 may acquire an ECG signal from the ECG data acquired by the ECG waveform acquisition unit 140. The ECG waveform analysis unit 150 may convert the ECG data into an analog signal to acquire an ECG signal. Also, the ECG waveform analysis unit 150 may extract multiple clinically important indicators from the acquired ECG signal.

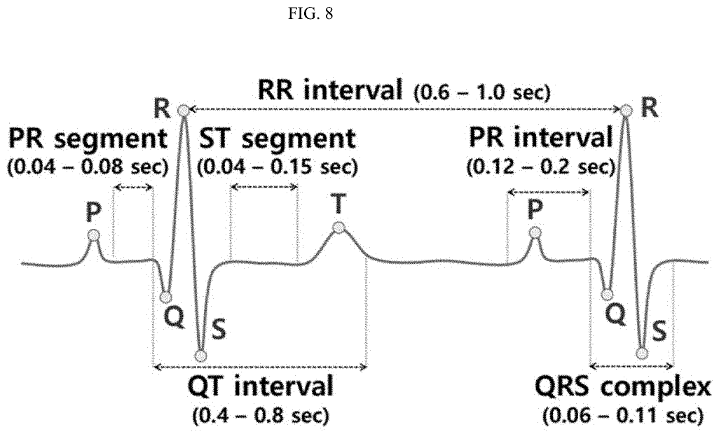

FIG. 8 is a diagram illustrating important clinical indicators corresponding to ECG signal waveforms.

As shown in FIG. 8, the ECG waveform analysis unit 150 may extract various clinical indicators (e.g., time stamps such as P, Q, R, S, T, RR interval, and QRS segment length) including five peaks P, Q, R, S, and T, from the ECG signal and output the extracted clinical indicators.

Here, a time stamp such as RR interval and QRS segment length refers to a time interval corresponding to the five peaks P, Q, R, S, and T of the ECG waveform, as shown in FIG. 8. In FIG. 8, time stamps for normal people with no heart disease are also displayed.

Here, the ECG waveform analysis unit 150 extracts multiple predetermined clinical indicators from the ECG data to easily determine the heart condition and may be omitted in some cases. Also, although it has been described above that the ECG waveform analysis unit 150 converts the ECG data into an analog signal to obtain an ECG signal and extracts a clinical indicator, the ECG waveform analysis unit 150 may be configured to extract a clinical indicator directly from the ECG data.

Also, the ECG waveform analysis unit 150 is also separately illustrated for convenience of description, and the ECG waveform analysis unit 150 may be included in the ECG waveform acquisition unit 140.

Conventional ECG measurement equipment of clinical grade is expensive equipment costing over $10,000 and up to $30,000. Even relatively inexpensive equipment costs more than $3,000, which is not a very affordable price for home use. On the other hand, the ECG monitoring device according to an embodiment may be implemented at a low cost of less than $100 by using a low-cost small vibration sensor, such as a geophone. Therefore, the ECG monitoring device may be placed in the home of each subject under observation without a great burden, and the ECG monitoring device may be placed in each hospital room in a large hospital and a small hospital. Thus, it is possible to regularly monitor heart anomalies. Therefore, it is possible to provide an early response to emergency situations.

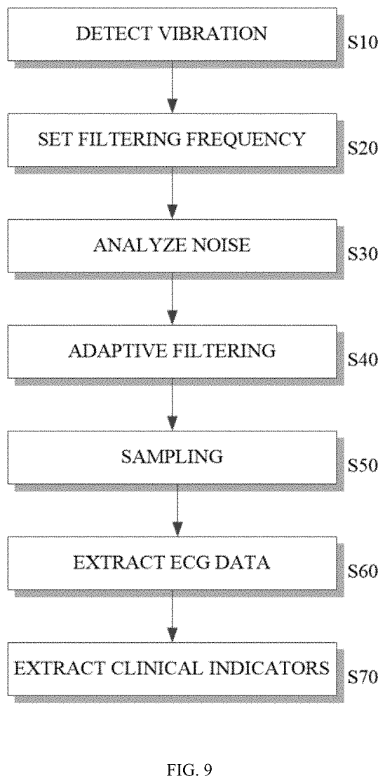

FIG. 9 illustrates an ECG monitoring method according to an embodiment of the present disclosure.

The ECG monitoring method according to an embodiment will be described below with reference to FIGS. 1 to 8. First, a vibration signal is acquired by detecting a vibration from a subject under observation in a non-contact and non-invasive manner through at least one vibration sensor attached to an instrument where the subject under observation is located (S10). In this case, the acquired vibration signal may contain vibrations due to breathing, movement, and the like of the subject under observation and noise caused by the vibration sensor itself and the surrounding environment along with the heart vibration of the subject under observation.

Also, while the subject under observation is not located in the instrument, a filtering frequency for removing noise is set by analyzing the vibration signal acquired in advance (S20). Here, the noise may include the intrinsic noise of the vibration sensor and the noise corresponding to the surrounding environment, and the intrinsic noise of the vibration sensor may be directly input and stored in the form of a user command or the like. However, a minimum frequency band capable of maintaining an SCG signal component may be preset to prevent an SCG signal component included in a vibration signal from being significantly damaged by the set filtering frequency. In some cases, the minimum frequency band for the SCG signal may be set as the filtering frequency without noise analysis.

When the filtering frequency is set, the SCG signal is acquired by filtering the vibration signal according to the set filtering frequency (S40). Here, the noise filtering may be performed by a combination of an LPF and an HPF. Then, the acquired SCG signal is sampled at a predetermined sampling rate and converted into a digital signal to acquire SCG data (S50).

Meanwhile, when the SCG signal is acquired, ECG data is extracted by estimating the pattern of the SCG signal using a pre-trained artificial neural network (S50). In this case, Bi-LSTM may be used as an artificial neural network, and accurate ECG data corresponding to a change in pattern of an SCG signal in a wide time interval may be extracted by using an artificial neural network in which two Bi-LSTM layers (Bi-LSTM layer-1 and Bi-LSTM layer-2) are stacked.

When the ECG data is extracted, multiple clinically important clinical indicators (e.g., time stamps such as P, Q, R, S, T, RR interval, and QRS segment length) are extracted from the extracted ECG data (S70).

Accordingly, with the electrocardiogram monitoring device and method according to an embodiment of the present disclosure, it is possible to extract the electrocardiogram of a subject under observation at low cost in a non-contact and non-invasive manner by attaching a small vibration sensor such as a geophone, instead of ECG measurement equipment, to a bed where the subject sleeps, etc. to detect vibrations. Therefore, it is possible to accurately monitor the electrocardiogram at low cost in each home where expensive ECG measuring equipment cannot be provided without causing inconvenience to a subject under observation or disturbing his or her sleep pattern, thus facilitating a response to emergency situations.

The method according to the present disclosure may be implemented as a computer program stored in a medium for execution by a computer. Here, the computer-readable medium may be any available medium accessible by a computer and may include any computer storage medium. The computer storage medium includes volatile and non-volatile media and discrete and integrated media which are implemented in any method or technique for storing information such as computer-readable instructions, data structures, program modules, or other data and may include a read-only memory (ROM), random-access memory (RAM), compact disc (CD)-ROM, digital versatile disc (DVD)-ROM, magnetic tapes, floppy disks, optical data storage devices, and the like.

While the present disclosure has been described with reference to an embodiment shown in the accompanying drawings, it should be understood by those skilled in the art that this embodiment is merely illustrative of the disclosure and that various modifications and equivalents may be made without departing from the spirit and scope of the invention.

Accordingly, the technical scope of the present disclosure should be determined only by the technical spirit of the appended claims.

What is claimed is:

1. An electrocardiogram (ECG) monitoring device comprising:
a vibration sensor unit configured to detect a vibration transferred through an instrument in a non-contact and non-invasive manner to acquire a vibration signal, the vibration sensor unit comprising at least one vibration sensor attached to the instrument where an object under observation is located;
a filter unit configured to receive the vibration signal, filter out a predetermined frequency band from the received vibration signal, and extract a seismocardiography (SCG) signal caused by a heart vibration of a subject under observation; and
an ECG waveform acquisition unit configured to estimate a pattern of the SCG signal and generate an ECG signal of a pattern corresponding to the pattern of the SCG signal, the ECG waveform acquisition unit comprising a pre-trained artificial neural network.

2. The ECG monitoring device of claim 1, wherein the filter unit comprises:
a first filter unit implemented as a low pass filter and configured to receive the vibration signal and filter the vibration signal to remove a frequency band of more than a first predetermined frequency; and
a second filter unit implemented as a high pass filter and configured to receive the filtered signal from the first filter unit and filter the received signal from the first filter unit to remove a frequency band of less than a second predetermined frequency.

3. The ECG monitoring device of claim 2, wherein the filter unit further comprises a noise analysis unit configured to set the first frequency and the second frequency according to at least one of intrinsic noise of the at least one vibration sensor or noise caused by an ambient environment of the instrument and provide the first frequency and the second frequency to the first filter unit and the second filter unit.

4. The ECG monitoring device of claim 3, wherein the noise analysis unit sets the first frequency and the second frequency according to a frequency of the vibration signal applied at a predetermined time when the subject under observation is not located in the instrument or a frequency detected from the vibration signal while a strength of a signal of a specific frequency band is less than or equal to a reference strength.

5. The ECG monitoring device of claim 3, wherein a frequency band of the intrinsic noise of the at least one vibration sensor is measured and stored in advance, and the noise analysis unit sets the first frequency and the second frequency according to the stored frequency band of the intrinsic noise.

6. The ECG monitoring device of claim 5, wherein
the at least one vibration sensor is implemented as a geophone, and
the noise analysis unit sets the first frequency to 30 Hz and sets the second frequency to 5 Hz.

7. The ECG monitoring device of claim 1, wherein the ECG waveform acquisition unit comprises:
a sampling unit configured to sample the SCG signal at a predetermined sampling rate to convert the SCG signal into SCG data; and
an ECG pattern estimation unit implemented as a pre-trained Bidirectional Long/Short-Term Memory (Bi-LSTM) neural network and configured to estimate a pattern change over time of the SCG data, which is time-series data, and acquire ECG data having a pattern corresponding to the estimated pattern change.

8. The ECG monitoring device of claim 7, wherein the ECG pattern estimation unit comprises:
a first Bi-LSTM layer comprising a forward layer and a backward layer each including multiple LSTM cells that receive SCG data included in a corresponding area while moving a sliding window of a predetermined size in chronological order with respect to the SCG data and extract features of the SCG data in a pre-trained manner and an activation layer configured to normalize outputs of the forward layer and the backward layer and output the normalized outputs;
a second Bi-LSTM layer comprising a forward layer and a backward layer each including multiple LSTM cells that receive an output of the first Bi-LSTM layer and extract features of the output of the first Bi-LSTM layer in a pre-trained manner and an activation layer configured to normalize outputs of the forward layer and the backward layer and output the normalized outputs; and
a regression layer configured to classify an output of the second Bi-LSTM layer as a corresponding value in a pre-trained manner and acquire the ECG data, the regression layer comprising multiple fully connected layers.

9. The ECG monitoring device of claim 8, wherein the ECG pattern estimation unit is trained by calculating, among learning data including multiple pieces of SCG data pre-acquired using a separate ECG measurement device and multiple pieces of ECG data mapped to the multiple pieces of SCG data, an error between ECG data acquired by receiving the multiple pieces of SCG data as an input and ECG data mapped in the learning data and backpropagating the calculated error.

10. The ECG monitoring device of claim 9, wherein
the ECG waveform acquisition unit further comprises a personal data storage unit configured to store personal data including multiple pieces of SCG data pre-acquired for the subject under observation using a separate ECG measurement device and multiple pieces of ECG data mapped to the multiple pieces of SCG data, and
the ECG pattern estimation unit is additionally trained using the personal data stored in the personal data storage unit.

11. The ECG monitoring device of claim 7, wherein the ECG waveform acquisition unit further comprises an ECG waveform analysis unit configured to receive the ECG data and convert the ECG data into an analog signal to acquire the ECG signal or analyze the ECG data to extract multiple predetermined clinical indicators.

12. An electrocardiogram (ECG) monitoring method comprising:
detecting a vibration transferred through an instrument in a non-contact and non-invasive manner to acquire a vibration signal using at least one vibration sensor attached to the instrument where an object under observation is located;
receiving the vibration signal, filtering the received vibration signal to remove a predetermined frequency band, and extracting a seismocardiography (SCG) signal caused by a heart vibration of the subject under observation; and
estimating a pattern of the SCG signal and generating an ECG signal of a pattern corresponding to the pattern of the SCG signal using a pre-trained artificial neural network.

13. The ECG monitoring method of claim 12, wherein the extracting the SCG signal comprises:
receiving the vibration signal, and low-pass-filtering the vibration signal to remove a frequency band of more than a first predetermined frequency; and
receiving the low-pass-filtered signal, and high-pass-filtering the received signal to remove a frequency band of less than a second predetermined frequency.

14. The ECG monitoring method of claim 13, wherein the extracting the SCG signal further comprises setting the first frequency and the second frequency according to a frequency of the vibration signal applied at a predetermined time when the subject under observation is not located in the instrument or a frequency detected from the vibration signal while the strength of a signal of a specific frequency band is less than or equal to a reference strength.

15. The ECG monitoring method of claim 13, wherein a frequency band of intrinsic noise of the at least one vibration sensor is measured and stored in advance, and the extracting the SCG signal further comprises setting the first frequency and the second frequency according to the stored frequency band of the intrinsic noise.

16. The ECG monitoring method of claim 12, wherein the generating the ECG signal comprises:
sampling the SCG signal at a predetermined sampling rate to convert the SCG signal into SCG data; and
estimating a pattern change over time of the SCG data, which is time-series data, using a pre-trained Bidirectional Long/Short-Term Memory (Bi-LSTM) neural network and acquiring ECG data having a pattern corresponding to the estimated pattern change.

17. The ECG monitoring method of claim 16, wherein the acquiring the ECG data comprises:
applying SCG data included in a corresponding area to a first Bi-LSTM layer including a forward layer and a backward layer each including multiple LSTM cells and an activation layer configured to normalize outputs of the forward layer and the backward layer and output the normalized outputs while moving a sliding window of a predetermined size in chronological order with respect to the SCG data, and extracting features of the SCG data in a pre-trained manner;
applying an output of the first Bi-LSTM layer to a second Bi-LSTM layer including a forward layer and a backward layer each including multiple LSTM cells and an activation layer configured to normalize outputs of the forward layer and the backward layer and output the normalized outputs and extracting features of the output of the first Bi-LSTM layer in a pre-trained manner; and
classifying the output of the second Bi-LSTM layer as a corresponding value using a regression layer including multiple fully connected layers, and extracting the ECG data.

18. The ECG monitoring method of claim 17, wherein
the Bi-LSTM neural network is pre-trained by a pre-performed training operation, and
the training operation comprises:
receiving, among learning data including multiple pieces of SCG data pre-acquired using a separate ECG measurement method and multiple pieces of ECG data mapped to the multiple pieces of SCG data, the multiple pieces of SCG data as an input and acquiring ECG data;
calculating an error between the acquired ECG data and ECG data mapped to the SCG data input from the learning data in a predetermined manner; and
backpropagating the calculated error.

19. The ECG monitoring method of claim 18, further comprising, after the training operation, a personalization operation in which the Bi-LSTM neural network is additionally trained using personal data including multiple pieces of SCG data pre-acquired for the subject under observation using a separate ECG measurement method and multiple pieces of ECG data mapped to the multiple pieces of SCG data.

20. The ECG monitoring method of claim 16, wherein the generating the ECG signal further comprises, after the acquiring the ECG data:
receiving the acquired ECG data and converting the ECG data into an analog signal to acquire the ECG signal; and
analyzing the ECG data to extract multiple predetermined clinical indicators.
